(19) United States
(12) Patent Application Publication
(10) Pub. No.: US 2013/0085201 A1
(43) Pub. Date: Apr. 4, 2013

(54) KETAL LACTONES AND STEREOSPECIFIC ADDUCTS OF OXOCARBOXYLIC KETALS WITH TRIMETHYLOL COMPOUNDS, POLYMERS CONTAINING THE SAME, METHODS OF MANUFACTURE, AND USES THEREOF

(71) Applicants: Sergey Selifonov, Plymouth, MN (US); Ning Zhou, Saint Paul, MN (US); Brian D. Mullen, Delano, MN (US)

(72) Inventors: Sergey Selifonov, Plymouth, MN (US); Ning Zhou, Saint Paul, MN (US); Brian D. Mullen, Delano, MN (US)

(73) Assignee: SEGETIS, INC., Golden Valley, MN (US)

(21) Appl. No.: 13/497,345

(22) PCT Filed: Sep. 27, 2010
(86) PCT No.: PCT/US10/50387

§ 371 Date: Nov. 5, 2012

Related U.S. Application Data
(60) Provisional application No. 61/246,102, filed on Sep. 26, 2009.

(51) Int. Cl. C07D 493/08; C08G 63/06; C08L 67/04; C07D 319/06; C07D 493/18

(52) U.S. Cl. CPC C07D 493/08; C07D 319/06; C07D 493/18; C08L 67/04; C08G 63/06
USPC 521/182; 549/267; 549/375; 525/415; 528/271; 524/599

(57) ABSTRACT

Ketal lactones of and methods for making such ketal lactones are disclosed. Also described are methods for making isolated cis- and trans-stereoisomers of hydroxyester ketals of oxocarboxylic acids and polymers having ketal units of such stereoisomers within the polymer backbone.

### FIELD

The invention relates generally to ketal compounds, and more specifically to ketal esters of oxocarboxylic acids, methods for their manufacture, and uses thereof.

### BACKGROUND

Many known chemical products such as surfactants, plasticizers, solvents, and polymers are currently manufactured from non-renewable, expensive, petroleum-derived or natural gas-derived feedstock compounds. High raw material costs and uncertainty of future supplies provide a need for the discovery and development of these and other chemical products that can be made from inexpensive, renewable biomass-derived feedstocks and by simple chemical methods. Using renewable resources as feedstocks for chemical processes will reduce the demand on non-renewable fossil fuels currently used in the chemical industry and reduce the overall production of carbon dioxide, the most notable greenhouse gas.

It is desirable to provide commonly used chemical products, such as surfactants, plasticizers, solvents, polymers, and the like from renewable feedstocks as a source of chemical building blocks. There is further a need for chemical building blocks that are a of chemically and thermally stable. It would be a still further advantage for the chemical building blocks to have multiple functionalities for subsequent reactions. Moreover, it is desirable to provide such materials by simple and/or and reproducible methods.

### SUMMARY

Novel ketal lactones of formula (1), specifically (1a), are disclosed:

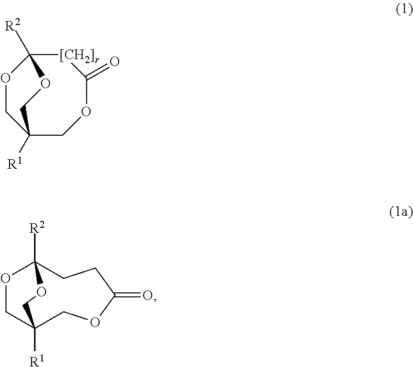

wherein r in formula (1) is 1-4 and each R¹ and R² is independently a C1-10 alkyl or C2-10 alkenyl.

In another embodiment, further disclosed are methods for making the isolated cis-hydroxyester ketal of formulae (2a):

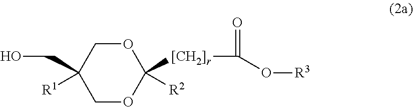

wherein R¹ and R² are as described above, and R³ is C1-6 alkyl, C2-6 alkenyl, C3-6 cycloalkyl, C5-6 cycloalkenyl, C6-12 aryl, C7-C13 arylalkylene, C2-10 alkyloxyalkylene, or C3-10 alkyloxyalkyleneoxyalkylene.

Further, in another embodiment, described is a polyester polymer comprising ketal units of formula (3):

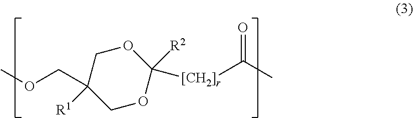

wherein r, R¹, and R² are as described above, and wherein, in one aspect, at least about 60 mol % of the ketal units of formula (3) are units of the cis-configuration of formula (3a):

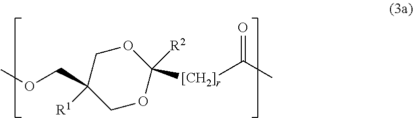

wherein r, R¹, and R² are as described above.

In another aspect, at least about 60 mol % of the ketal units of formula (3) are units of the trans-configuration of formula (3b):

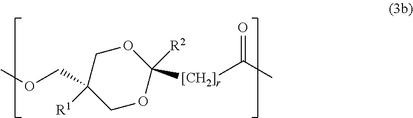

wherein r, R¹, and R² are as described above.

Also disclosed is a compound of formula (4), specifically (4a):

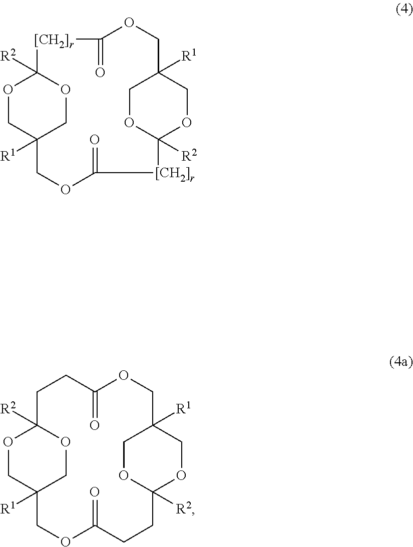

wherein each R¹ and each R² can be the same or different, and are as defined above.

Methods for the manufacture and uses of the foregoing compounds are also described.

### DETAILED DESCRIPTION

Disclosed herein are compounds based on the oxocarboxylic ketals, the compounds being useful as chemical products such as surfactants, plasticizers, solvents, polymers, and the like that can be produced from renewable feedstocks. The chemical compounds have multiple functionalities for subsequent reactions. Moreover, such materials can be obtained by simple and/or and reproducible methods.

In an embodiment, a novel ketal lactone is of formula (1):

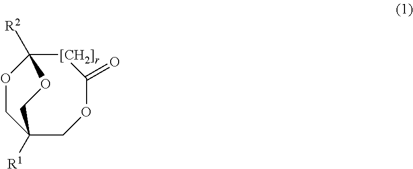

wherein each R¹ and R² is independently a C1-10 alkyl or C2-10 alkenyl, and r is 1-4. In specific embodiment, each R¹ and R² is independently a C1-6 alkyl or C2-6 alkenyl, and r is 1-4. Still more specifically, each R¹ and R² is independently a C1-3 alkyl and r is 1-3.

A specific embodiment of the ketal lactone of formula (1) is the lactone of formula (1a):

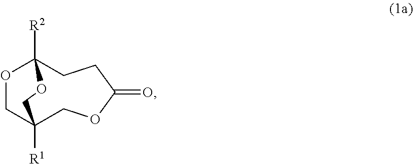

wherein each R¹ and R² is independently a C1-10 alkyl or C2-10 alkenyl. Specifically each R¹ and R² is independently a C1-6 alkyl or C2-6 alkenyl, and still more specifically, each R¹ and R² is independently a C1-3 alkyl.

Another specific embodiment of the ketal lactone of formula (1) is the levulinic lactone of formula (1b):

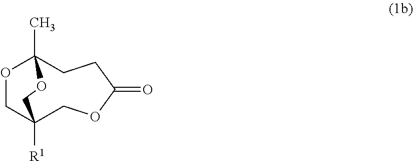

wherein R¹ is a C1-10 alkyl or C2-10 alkenyl, specifically a C1-6 alkyl or C2-6 alkenyl, still more specifically methyl or ethyl.

Also disclosed herein are certain stereospecific isomers of hydroxyester ketals. Certain hydroxyester ketals, e.g., of formula (2) are known in the art:

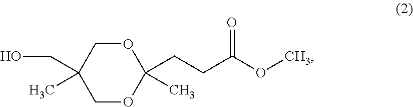 [SMILES: COC(=O)CCC1(C)OCC(C)(CO)CO1]

However, known methods for making compounds of formula (2) are cumbersome and do not appear to be stereoselective, such that the isolated stereoisomers of compound (2) do not appear to be known in the art. Thus, also described herein are the cis- and trans-hydroxyester ketals having formulae (2a) and 2(b):

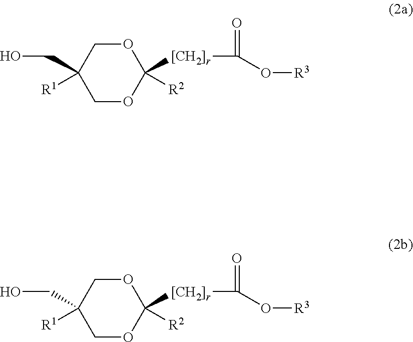

wherein each R¹ and R² is independently a C1-10 alkyl or C2-10 alkenyl, r is 1-4, and R³ is C1-6 alkyl, C2-6 alkenyl, C3-6 cycloalkyl, C5-6 cycloalkenyl, C6-12 aryl, C7-C13 arylalkylene, C2-10 alkyloxyalkylene, or 3-10 alkyloxyalkyleneoxyalkylene. In a specific embodiment, each R¹ and R² is independently a C1-6 alkyl or C2-6 alkenyl, r is 1-4, and R³ is C1-6 alkyl, C6-12 aryl, C7-C13 arylalkylene, C2-10 alkyloxyalkylene, or 3-10 alkyloxyalkyleneoxyalkylene. Still more specifically, each R¹ and R² can be independently a C1-3 alkyl and r is 1-3, and R³ is C1-4 alkyl, C6-12 aryl, C7 arylalkylene, C2-10 or alkyloxyalkylene, or 3-10 alkyloxyalkyleneoxyalkylene.

Another specific embodiment of the hydroxyester ketals of formula (2a) and (2b) are the hydroxyester ketals of formulae (2c) and (2d):

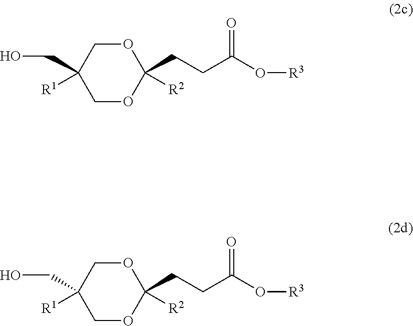

wherein each R¹ and R² is independently a C1-10 alkyl or C2-10 alkenyl and R³ is C1-6 alkyl, C2-6 alkenyl, C3-6 cycloalkyl, C5-6 cycloalkenyl, C6-12 aryl, C7-C13 arylalkylene, C2-10 alkyloxyalkylene, or C3-10 alkyloxyalkyleneoxyalkylene. Specifically each R¹ and R² can be independently a C1-6 alkyl or C2-6 alkenyl and R³ can be a C1-6 alkyl, C6-12 aryl, C7-C13 arylalkylene, C2-10 alkyloxyalkylene, or C3-10 alkyloxyalkyleneoxyalkylene. Still more specifically, each R¹ and R² can be independently a C1-3 alkyl and R³ can be a C1-6 alkyl, C7 arylalkylene, C2-10 alkyloxyalkylene, or C3-10 alkyloxyalkyleneoxyalkylene.

Another specific embodiment of hydroxyester ketals of formula (2a) and (2b) are the hydroxyester ketals of formulae (2e) and (2f):

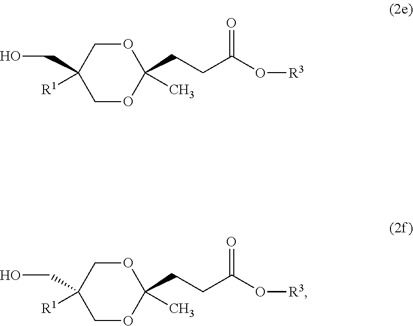

wherein R¹ is a C1-10 alkyl, and R³ is C1-6 alkyl, C2-6 alkenyl, C3-6 cycloalkyl, C5-6 cycloalkenyl, C6-12 aryl, C7-C13 arylalkylene, C2-10 alkyloxyalkylene, or C3-10 alkyloxyalkyleneoxyalkylene. Specifically R¹ can be a C1-6 alkyl, and R³ can be a C1-6 alkyl, C6-12 aryl, C7-C13 arylalkylene, C2-10 alkyloxyalkylene, or C3-10 alkyloxyalkyleneoxyalkylene. Still more specifically, R¹ can be a methyl or ethyl and R³ can be a C1-6 alkyl, C7 arylalkylene, C2-10 alkyloxyalkylene, or C3-10 alkyloxyalkyleneoxyalkylene.

Further, compositions comprising a combination of the cis isomers of formulas (2a), (2c), and/or (2e) and the trans isomers of formulas (2b), (2d), and/or (2f) can be prepared. In an embodiment, the ratio of the quantity of the cis isomers of formulas (2a), (2c), and/or (2e) with respect to the quantity of a trans isomers of formulas (2b), (2d), and/or (2f) is in a range between 0 and 0.35, specifically in a range from 0.001 to 0.25. Similarly, such compositions can comprise a ratio of the quantity of the trans isomers of formulas (2b), (2d), and/or (2f) with respect to the quantity of the cis isomers of formulas (2a), (2c), and/or (2e) in a range between 0 and 0.35, specifically in a range from 0.001 to 0.25.

Also disclosed is a compound of formula (4):

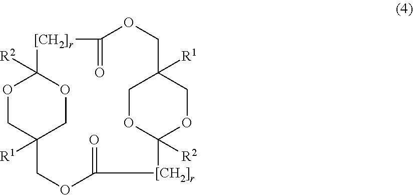

wherein each R¹ and R² is independently a C1-10 alkyl or C2-10 alkenyl and r=1-4. Specifically each R¹ and R² is independently a C1-6 alkyl or C2-6 alkenyl and r=1-4, and still more specifically, each R¹ and R² is independently a C1-3 alkyl and r=1-3.

Another specific embodiment of the compound of formula (4) is the compound of formula (4a):

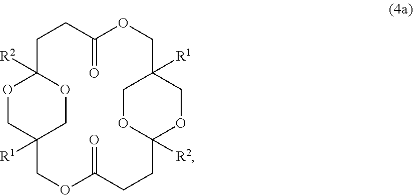

wherein each R¹ and R² is independently a C1-10 alkyl or C2-10 alkenyl. Specifically each R¹ and R² is independently a C1-6 alkyl or C2-6 alkenyl, and still more specifically, each R¹ and R² is independently a C1-3 alkyl.

Another specific embodiment of the ketal lactone of formula (1) is the levulinic lactone of formula (1b):

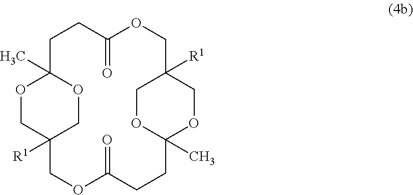

wherein R¹ is a C1-10 alkyl or C2-10 alkenyl, specifically a C1-6 alkyl or C2-6 alkenyl, still more specifically methyl or ethyl.

In an embodiment, hydroxyester ketals of formula (2a) and (2b), specifically (2b) and (2d), more specifically (2e) and (2f) are obtained by enantiomeric resolution of a combination of the cis- and trans-isomers. The mixtures of cis- and trans-isomers can be obtained by means known in the art, for example acid-catalyzed condensation of an oxocarboxylic acid of formula (5)

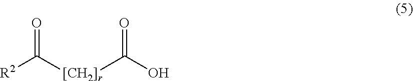

wherein R² is a C1-10 alkyl or C2-10 alkenyl and r is 1-4 with an alcohol of formula (6)

(HO)ₛ—R³ (6)

wherein R³ is C1-6 alkyl, C2-6 alkenyl, C3-6 cycloalkyl, C5-6 cycloalkenyl, C6-12 aryl, C7-C13 arylalkylene, C2-10 alkyloxyalkylene, or C3-10 alkyloxyalkyleneoxyalkylene. In a specific embodiment, R³ in formula (7) is a C1-6 alkyl, e.g., methyl, ethyl, or n-butyl. Alternatively, R³ in formula (7) can be an enantiomeric resolving agent. Methods for enantiomeric resolution of cis- and trans-isomers are known in the art, and include chiral chromatography, selective crystallization, and the like. For example, a combination of a cis-isomer compound of formula (2a), specifically (2c), and more specifically (2e) and a trans-isomer compound of formula (2b), specifically (2d), more specifically (2f), comprising either isomer in excess of 60% by weight, can be prepared by fractional distillation of a cis/trans mixture of the stereoisomers comprising approximately equal amounts of cis- and trans-isomers. In another aspect, the combination of a cis-isomer compound of formula (2a), specifically (2c), and more specifically (2e) and a trans-isomer compound of formula (2b), specifically (2d), more specifically (2f), can comprise either isomer in excess of 80% by weight. Further in another aspect a cis-isomer compound of formula (2a), specifically (2c), and more specifically (2e) and a trans-isomer compound of formula (2b), specifically (2d), more specifically (2f) can comprise either isomer in excess of 90% by weight.

Once obtained, the cis- and trans-isomers, individually or in a specific ratio, can be polymerized to obtain polymers comprising predominantly one or the other isomer, for example polymers comprising the cis-isomer units of formula (3a):

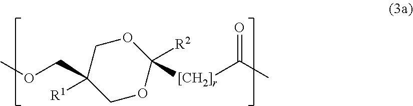

wherein each R¹ and R² is independently a C1-10 alkyl or C2-10 alkenyl, and r is 1-4. Such polymers can comprise at least 2, specifically 2-1,000 cis-isomer ketal units, more specifically 3-500, still more specifically 3-250, yet more specifically 4-100, even more specifically 5-50, and still more specifically 10-40 cis-isomer ketal units. It is also possible to have 2-100, 2-50, 2-35, 2-20, and 2-10 cis-isomer ketal units. In still another embodiment, the polymers have 20-1,000, 50-1,000,200-1,000, or 400-1,000 cis-isomer ketal units.

Moreover, the polyester comprising units of formula (3a) can have a weight average molecular weight from 400 to 10,000 Daltons. In an embodiment, each R¹ and R² is independently a C1-6 alkyl or C2-6 alkenyl, and r is 1-4. In another embodiment, each R¹ and R² is independently a C1-3 alkyl, and r is 1-3.

Specifically the cis-isomer units are of formula (3c):

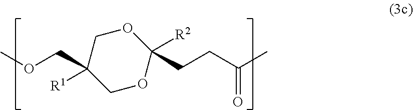

wherein each R¹ and R² is independently a C1-10 alkyl or C2-10 alkenyl, and r is 1-4. Such polymers can comprise at least 2, specifically 2-1,000 cis-isomer ketal units, more specifically 3-500, still more specifically 3-250, yet more specifically 4-100, even more specifically 5-50, and still more specifically 10-40 cis-isomer ketal units. It is also possible to have 2-100, 2-50, 2-35, 2-20, and 2-10 cis-isomer ketal units. In still another embodiment, the polymers have 20-1,000, 50-1,000,200-1,000, or 400-1,000 cis-isomer ketal units. Moreover, the polyester comprising units of formula (3c) can have a weight average molecular weight from 400 to 10,000 Daltons. In an embodiment, each R¹ and R² is independently a C1-6 alkyl or C2-6 alkenyl. In another embodiment, each R¹ and R² is independently a C1-3 alkyl.

Still more specifically, the cis-isomer units can be of formula (3e):

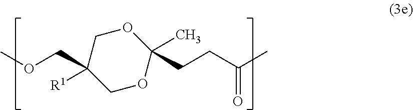

wherein R¹ is a C1-10 alkyl or C2-10 alkenyl, specifically a C1-6 alkyl or C2-6 alkenyl, still more specifically methyl or ethyl. Such polymers can comprise at least 2, specifically 2-1,000 cis-isomer ketal units, more specifically 3-500, still more specifically 3-250, yet more specifically 4-100, even more specifically 5-50, and still more specifically 10-40 cis-isomer ketal units. It is also possible to have 2-100, 2-50, 2-35, 2-20, and 2-10 cis-isomer ketal units. In still another embodiment, the polymers have 20-1,000, 50-1,000,200-1,000, or 400-1,000 cis-isomer ketal units. Moreover, the polyester comprising units of formula (3e) can have a weight average molecular weight from 400 to 10,000 Daltons.

The cis- and trans-isomers, individually or in a specific ratio, can be polymerized to obtain polymers comprising predominantly trans-isomer units as in formula (3b):

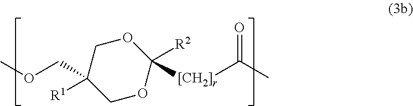

wherein each R¹ and R² is independently a C1-10 alkyl or C2-10 alkenyl, and r is 1-4. Such polymers can comprise at least 2, specifically 2-1,000 trans-isomer ketal units, more specifically 3-500, still more specifically 3-250, yet more specifically 4-100, even more specifically 5-50, and still more specifically 10-40 trans-isomer ketal units. It is also possible to have 2-100, 2-50, 2-35, 2-20, and 2-10 trans-isomer ketal units. In still another embodiment, the polymers have 20-1,000, 50-1,000,200-1,000, or 400-1,000 trans-isomer ketal units. Moreover, the polyester comprising units of formula (3b) can have a weight average molecular weight from 400 to 10,000 Daltons. In an embodiment, each R¹ and R² is independently a C1-6 alkyl or C2-6 alkenyl, and r is 1-4. In another embodiment, each R¹ and R² is independently a C1-3 alkyl, and r is 1-3.

In a specific embodiment, the polymer comprises trans-isomer units as in formula (3d)

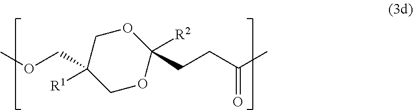

wherein each R¹ and R² is independently a C1-10 alkyl or C2-10 alkenyl, and r is 1-4. Such polymers can comprise at least 2, specifically 2-1,000 trans-isomer ketal units, more specifically 3-500, still more specifically 3-250, yet more specifically 4-100, even more specifically 5-50, and still more specifically 10-40 trans-isomer ketal units. It is also possible to have 2-100, 2-50, 2-35, 2-20, and 2-10 trans-isomer ketal units. In still another embodiment, the polymers have 20-1,000, 50-1,000,200-1,000, or 400-1,000 trans-isomer ketal units. Moreover, the polyester comprising units of formula (3d) can have a weight average molecular weight from 400 to 10,000 Daltons. In an embodiment, each R¹ and R² is independently a C1-6 alkyl or C2-6 alkenyl. In another embodiment, each R¹ and R² is independently a C1-3 alkyl.

In another specific embodiment, the polymer comprises trans-isomer units of formula (3f)

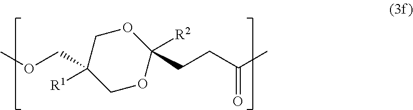

wherein R¹ is a C1-10 alkyl or C2-10 alkenyl, specifically a C1-6 alkyl or C2-6 alkenyl, still more specifically methyl or ethyl. Such polymers can comprise at least 2, specifically 2-1,000 trans-isomer ketal units, more specifically 3-500, still more specifically 3-250, yet more specifically 4-100, even more specifically 5-50, and still more specifically 10-40 trans-isomer ketal units. It is also possible to have 2-100, 2-50, 2-35, 2-20, and 2-10 trans-isomer ketal units. In still another embodiment, the polymers have 20-1,000, 50-1,000,200-1,000, or 400-1,000 trans-isomer ketal units. Moreover, the polyester comprising units of formula (3f) can have a weight average molecular weight from 400 to 10,000 Daltons.

In another aspect, the residual polymer comprises about 65 mol % to about 90 mol % of the trans-isomer units of formula (3b), specifically (3d), more specifically (3f), and about 10 to about 35 mol % of the cis-isomer units of formula (3a), specifically (3c), more specifically (3e). In another aspect, the residual polymer comprises about 80 mol % to about 90 mol % of the trans-isomer units of formula (3b), specifically (3d), more specifically (3f), and about 10 to about 20 mol % of the cis-isomer units of formula (3a), specifically (3c), more specifically (3e). In another aspect, the residual polymer comprises about 90 mol % to about 99 mol % of the trans-isomer units of formula (3b), specifically (3d), more specifically (3f), and about 1 to about 10 mol % of the cis-isomer units of formula (3a), specifically (3c), more specifically (3e).

Polymerization can occur in the presence of an acid catalyst under conditions allowing for removal of the water to form a polymer with ester and ketal groups. The acid catalyst can be either a Lewis or Brønsted-Lowry acid, for example strong protic acid catalysts, e.g., Brønsted-Lowry acids that have a Ka of 55 or greater. Examples of strong protic acid catalysts include sulfuric acid, arylsulfonic acids, and hydrates thereof such as p-toluenesulfonic acid monohydrate, methane sulfonic acid, camphor sulfonic acid, dodecyl benzene sulfonic acid, perchloric acid, hydrobromic acid, and hydrochloric acid. In other embodiments, weak protic acid catalysts, e.g., having a Ka of less than 55, can be used, for example phosphoric acid, orthophosphoric acid, polyphosphoric acid, and sulfamic acid. Aprotic (Lewis acid) catalysts can include, for example, titanium tetrachloride, aluminum trichloride, and boron trifluoride. A combination comprising any one or more of the foregoing acid catalysts can be used. In some embodiments, the method employs a substantially nonvolatile acid catalyst such that the acid does not transfer into the distillate, such as sulfuric or sulfamic acid. In an exemplary embodiment, the homogenous catalyst is camphor sulfonic acid.

Instead of, or in addition to the homogenous acid catalyst, a heterogenous acid catalyst can be used, where the acid catalyst is incorporated into, onto, or covalently bound to, a solid support material such as resin beads, membranes, porous carbon particles, zeolite materials, and other solid supports. Many commercially available resin-based acid catalysts are sold as ion exchange resins. One type of useful ion exchange resin is a sulfonated polystyrene/divinyl benzene resin, which supplies active sulfonic acid groups. Other commercial ion exchange resins include LEWATIT® ion exchange resins sold by the Lanxess Company of Pittsburgh, Pa.; DOWEX™ ion exchange resins sold by the Dow Company of Midland, Mich.; and AMBERLITE® and AMBERLYST® ion exchange resins sold by the Rohm and Haas Company of Philadelphia, Pa. In embodiments, AMBERLYST® 15 is used. In these embodiments, the resin based catalyst is washed with an alcohol, such as methanol or ethanol, and then dried prior to use. In use, the heterogenous catalysts are added to a reaction mixture, thereby providing a nonvolatile source of acid protons for catalyzing the reactions. The heterogenous catalysts can be packed into columns and the reactions carried out therein. As the reagents elute through the column, the reaction is catalyzed and the eluted products are free of acid. In other embodiments, the heterogenous catalyst is slurried in a pot containing the reagents, the reaction is carried out, and the resulting reaction products filtered or distilled directly from the resin, leaving an acid-free material.

The reaction is carried out typically in the presence of 0.0001 to 0.1 molar percent of an acid catalyst. Non-limiting examples of catalysts include sulfuric acid, alkyl or aryl or arylalkylenesulfonic acids, or heterogenous, porous or non-porous sulfonated polymers such as strongly acidic ion exchange resins known in the art. The reaction is carried out under conditions wherein the oxocarboxylic acid (5) and trimethylol compound (7) are combined in a molar ratio of about 1:1. Either of the two reactants can be used in excess. However, in another aspect, the ratio of the trimethylol compound (7) and the oxocarboxylic acid (5) are combined in a molar ratio from 0.8 to 1.2. The reaction is carried out until about 2 moles of water have been distilled out per each mol of oxocarboxylic acid.

Thus, isolated compounds (2a-f) are useful to prepare a variety of polymers comprising predominantly or substantially all cis-isomer units of formula (3a), (3c), or 3(e) or trans-isomer units of formula (3b), (3d), or (3f). Such polymers can have a total of 2-1,000 units, more specifically from 3-500 units, still more specifically from 3-250 units, yet more specifically 4-100 total units, even more specifically 5-50 total units, and still more specifically 10-40 total units. It is also possible to have 2-100 total units, 2-50 total units, 2-35 total units, 2-20 total units, and 2-10 total units. In still another embodiment, such polymers can have 20-1,000 total units, 50-1,000 total units, 200-1,000 total units, or 400-1,000 total units. Depending on the stereochemistry of the starting materials, the polymers can have more than 60 mole %, more than 65 mole %, more than 75 mole %, more than 85 mole %, more than 90 mole %, more than 95 mole %, or more than 95 mol % cis-isomers. Alternatively, the polymers can have more than 60 mole %, more than 65 mole %, more than 75 mole %, more than 85 mole % and up to 90 mole % trans-isomers.

The polymers are polyesters that can have a variety of end groups, including hydroxy groups, carboxyl groups, and/or different alkoxycarbonyl groups as shown in formula (8):

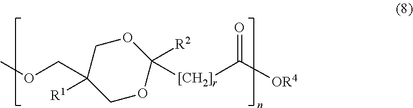

wherein r, n, R¹, and R² are as described in formula (3). It is to be understood that the stereochemistry of the polymer of formula (8) can vary and will depend on the stereochemistry of the starting materials. R⁴ is hydrogen or a moiety derived from a C1-10 monohydric or polyhydric alcohol of formula (9)

(HO)ₛ—R⁴ (9)

wherein s is 1-6 and R⁴ is a C1-10 hydrocarbon, for example, a C1-10 straight, branched-chain, or cyclic aliphatic group, a C2-6 straight, branched-chain, or cyclic aliphatic group having 1-2 double bonds, a C6-10 cyclic aromatic group, a C2-10 alkyloxyalkylene, or a C3-10 alkyloxyalkyleneoxyalkylene wherein each of the foregoing can be unsubstituted or substituted with 1-2 hydroxy groups, 1-2 (C1-3alkyl)carbonyl groups, 1-2 (C1-6)alkylcarbonyl groups, 1-2 (meth)acryloyl groups, or a combination thereof. The terminal groups R⁴ are provided by including in the polymerization mixture a suitable quantity of the monohydric or polyhydric alcohol of formula (9). Of course, the foregoing R⁴ groups include moieties of the formula R¹C(CH₂OH)₂(CH₂O—) wherein R¹ is as defined in formula (3). Alternatively, an ester interchange of the polymer can be conducted in the presence of a compound of formula (9) to provide R⁴. The polyester can contain two or more hydroxy groups, or a combination of hydroxy groups and alkoxycarbonyl groups. In an embodiment the monohydric or polyhydric alcohol can provide additional functionality to the polymer. For example, addition of an amount of (meth)allyl alcohol to the polymerization mixture, derivatization of the carboxyl group of the polymer, or ester interchange of the polymer with (meth)allyl alcohol provides a (meth)allyl ester terminal groups that can subsequently be used for derivatization or polymerization. Such polymers are useful for preparing a variety of adhesives, coatings, and thermoset articles.

An alternative route to the cis-isomers (2a), specifically (2c), more specifically (2e) proceeds first through a polyester comprising ketal ester units of formula (3):

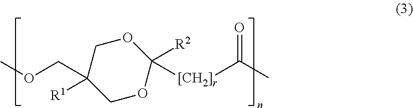

wherein each R¹ and R² is independently a C1-10 alkyl or C2-10 alkenyl, r is 1-4, and n is greater than 2, specifically 2-1,000, more specifically 3-500, still more specifically 3-250, yet more specifically 4-100, even more specifically 5-50, and still more specifically 10-40. It is also possible to have n=2-100, 2-50, 2-35, 2-20, and 2-10. In still another embodiment, n=20-1,000, 50-1,000,200-1,000, or 400-1,000. Moreover, the polyester comprising units of formula (3) can have a weight average molecular weight from 400 to 10,000 Daltons.

Polyesters comprising units of formula (3) can be obtained by reaction of an oxocarboxylic acid of formula (5)

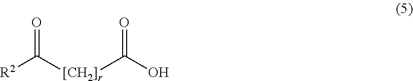

wherein R² is a C1-10 alkyl or C2-10 alkenyl and r is 1-4, with a trimethylol compound of formula (7):

R¹—C(CH₂OH)₃ (7)

wherein R¹ is a C1-10 alkyl or C2-10 alkenyl under acidic polymerization conditions as described above.

The polyester of formula (3) is then depolymerized by sufficient heating under reduced pressure, typically at temperatures between 100° C. and 250° C., in the presence of a suitable catalyst, for example a protonic acid, to provide a distillate comprising predominantly a ketal lactone of formula (1), specifically (1a) more specifically (1b), and optionally, a compound of formula (4), specifically (4a) more specifically (4b). The distillate can also optionally contain various quantities of each the starting compounds, or angelica lactone. The distillation is typically carried out until substantially all polymer or majority of the polymer has been depolymerized to compounds (1) and/or (4). Moreover, a partial distillation can be carried out to depolymerize and distill a smaller quantity of the compounds (1) and (4). Under acid catalyzed conditions, ketals of cis and trans stereochemistry equilibrate. However, only the cis-isomer forms the cyclic ketal lactone of formula (1).

Then, either of compounds (1), (4) can be transesterified to yield a cis-isomer of the hydroxyester of formula (2a), specifically (2b), more specifically (2c) in the presence of a transesterification catalyst. Transesterification is typically carried out under base-catalyzed conditions using alkali or alkali earth metal alkoxides, hydroxides or alkoxides of tin or titanium, typically in the presence of sufficient quantity of alcohol of formula R³—OH, wherein R³ is as defined in formulas (2a), specifically (2c), and more specifically (2e).

The compounds obtained by the transesterification can be further purified by distillation, typically under reduced pressure, in a batch mode, or using a continuous distillation by falling film, wiped film, spinning film or other distillation methods known in the art.

Transesterification of (1) and/or (4) accordingly provides an alternative method for obtaining hydroxyester of formula (2a), specifically (2b), more specifically (2c) wherein R³ is as described above. Transesterification of compounds (1) and/or (4) is a particularly effective method for achieving stereoselectively pure cis-isomers of formulas (2a), specifically (2b), more specifically (2c). Such compositions can comprise greater than 90 mole % cis-isomer, specifically greater than 95 mol %, and more specifically greater than 99 mole % of cis-isomer. Alternatively, in still another aspect, a combination of a cis-isomer compound of formula (2a), specifically (2c), and more specifically (2e) and a trans-isomer compound of formula (2b), specifically (2d), more specifically (2f) can comprise the cis-isomer in excess of 90% by weight, specifically in excess of 95% by weight, and more specifically in excess of 99% by weight.

In addition to the polymers described above, isolated compounds of formula (1), specifically (1a), more specifically (1b), compounds of formulas (2a-f), and compounds of formulas (4), specifically (4a), and more specifically (4b) can be used in the manufacture of polyester ketal copolymers with a variety of other monomers capable of forming ester bonds. Such copolymers can contain the foregoing amounts of cis- and trans-isomer.

Representative monomers that can be copolymerized include, but are not limited to, C1-36 aliphatic or C6-36 aromatic dicarboxylic or tricarboxylic acids and their reactive derivatives, e.g., the corresponding diaryl esters, anhydrides, salts, acid chlorides, and acid bromides. Representative acids include the ortho-, meta-, and para-isomers of phthalic acid, 2,6-naphthalene dicarboxylic acid, 1,2,4-benzene-tricarboxylic acid, adipic acid, succinic acid, glutaric acid, citric acid, itaconic acid, mesaconic acid, 2-methylsuccinic acid, azelaic acid, sebacic acid, and the like and a combination comprising a of the foregoing acids.

Other monomers can be copolymerized include polyhydric alcohols having from 2 to 6 hydroxyl groups, and their reactive derivatives, such as the corresponding C1-3 dialkyl esters, diaryl esters, and the like. Representative polyols can have from 2 to 36 carbon atoms, and include ethylene glycol, diethylene glycol, triethylene glycol, 1,2-propane diol, 1,3-propane diol, 1,2-butane diol, 1,3-butane diol, 1,4-butane diol, 1,4-butene diol, hexamethylene diol, sorbitol, xylitol, mannitol, erythritol, arabitol, pentaerythritol, di-pentaerythritol, trimethylol propane, trimethylol ethane, 2-methyl-1,3-propanediol, and the like, and a combination comprising a of the foregoing polyols.

Still other monomers that can be can be copolymerized include hydroxy-carboxylic acids, and their corresponding esters and lactones, for example lactic acid, glycolic acid, 3-hydroxyalkanoic acid, ricinoleic acids, lactide, glycolide bis-lactone, 1,4-dioxan-2-one, 1,4-dioxan-2-ones with optional C1-6alkyl or C6-12 aryl substituents at 3, 5, and 6 positions, 2-oxepanone, dioxephanone monomer, epsilon-caprolactone, trimethylene carbonate, dimethylene carbonate monomers, and the like, and a combination comprising a of the foregoing. A specific hydroxy-carboxylic acid that can be used is of formula (10):

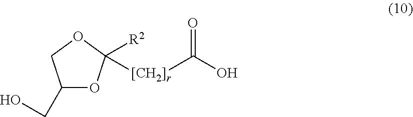

wherein r is 1-4 and R² is a C1-10 alkyl or C2-10 alkenyl. In a specific embodiment, R² is methyl and r=2.

Still other monomers that can be can be copolymerized oligomers and polymers with at least two terminal groups that can be hydroxyl and/or carboxyl groups. Such monomers include hydroxyl-terminated linear or branched polyethers, such as C6-36 polyethylene glycol, polypropylene glycol, polyepoxides of alpha-olefins, polyethoxylated polyhydric alcohols, polyethers derived from 1,3-propane diol having formula (—CH₂—CH₂—CH₂—O—)ₘ, wherein m is an integer from 2 to 12, and a combination comprising a of the foregoing; polyester polyols, that is, polyesters terminated with two or more hydroxyl groups; and polyurethane polyols, that is, polyurethanes terminated with two or more hydroxyl groups. Other hydroxyl- and/or carboxyl-terminated monomers of this type include polysaccharides, poly(3-hydroxyalkanoate), polylactate, polyglycolide, poly(co-lactate/glycolate), and the like, or a combination comprising of the foregoing. The foregoing oligomers and polymers, when used as monomers, can have from 2-1,000 units, specifically from 3 to 500 units, more specifically from 5 to 100 units, or from 10 to 50 units.

Primary and secondary amino compounds can be used to provide mixed polyester-polyamides, said amino compound optionally having one or more hydroxyl, carboxyl, ester, carboxamide, N-substituted carboxamide or ether groups.

Accordingly, further disclosed herein are polyester ketal copolymers comprising units of formula (3a-f) and a other polyester unit derived from a polyhydric alcohol having from 2 to 6 hydroxyl groups; a other unit derived from a C1-36 aliphatic or C6-36 aromatic dicarboxylic or tricarboxylic acids and their reactive derivatives; a other unit derived from a hydroxylated carboxylic acid and their corresponding esters and lactones; least one other unit derived from a hydroxyl-terminated linear or branched polyether; a other unit derived from a polyester polyol; a other unit derived from a polyurethane polyol; a other unit derived from a polysaccharide; a other unit derived from a poly(3-hydroxyalkanoate); a other unit derived from a polylactate; a other unit derived from a polyglycolide; a other unit derived from a poly(co-lactate/glycolate); or a combination thereof. Also disclosed are polyester-polyamides comprising units of formula (3a-f) and another unit derived from an amino compound having one or more amino groups, said amino compound optionally having one or more hydroxyl, carboxyl, ester, carboxamide, N-substituted carboxamide or ether groups.

The polyester ketal copolymers can have 2-1,000 units, more specifically from 3-500 units, still more specifically from 3-250 units, yet more specifically 4-100 units, even more specifically 5-50 units, and still more specifically 10-40 units. It is also possible to have 2-100 units, 2-50 units, 2-35 units, 2-20 units, and 2-10 units. In still another embodiment, such polymers can have 20-1,000 units, 50-1,000 units, 200-1,000 units, or 400-1,000 units. The ratio of ketal units of formulas (3a-f) to comonomer units can vary widely depending on the desired properties of the copolymer, and can be, for example, 1:99 to 99:1, specifically 10:90 to 90:10, more specifically 20:80 to 80:20, still more specifically 30:70 to 70:30, and yet more specifically 40:60 to 60:40.

The polyester ketal copolymers can be obtained by methods well known to those skilled in the art, including, for example, interfacial polymerization, melt-process condensation, solution phase condensation, and transesterification polymerization. Such polyesters are typically obtained through the condensation or ester interchange polymerization of the diol or diol equivalent component with the diacid or diacid chemical equivalent component. Methods for making polyesters and the use of polyesters in thermoplastic molding compositions are known in the art. Conventional polycondensation procedures are described, for example, in U.S. Pat. Nos. 5,367,011 and 5,411,999. The condensation reaction can be facilitated by the use of a catalyst, with the choice of catalyst being determined by the nature of the reactants. The various catalysts are known in the art.

The foregoing polymers can be useful in a variety of applications, for example as adhesives, coatings, sealants, and in the manufacture of articles. The polymers can be formed into articles using techniques known in the art, for example extruding, molding, stamping, thermoforming, casting, calendaring, laminating, and the like. The polymers can be combined with other polymers to provide blends, mixtures, alloys, and the like, and optionally further formulated with polymer additives such as reinforcing or particulate fillers, antioxidants, mold release agents, flame retardants, and the like. The nature of the other polymers and additives, if any will depend on the end use of the articles, for example fabrication of fibers, surgical instruments, stents, implants, prostheses, drug delivery vehicles, packaging (inluding food packaging),

The polyester ketal polymers obtained by polymerization or co-polymerization of any of the compounds of formula (1), specifically (1a), more specifically (1b), compounds of formulas (2a-b), specifically (2c-d), more specifically (2e-f), and compounds of formulas (4), specifically (4a), and more specifically (4b) with one or more of the above-described comonomers can be obtained as linear or branched copolymers. It is possible to obtain a branched polyester using a branching agent, for example, a glycol having three or more hydroxyl groups or a trifunctional or multifunctional carboxylic acid has been incorporated into the reaction mixture. When a branching agent or comonomer is used, the polyester ketal copolymers can be terminated with multiple hydroxyl groups, for example two to six hydroxy groups.

The hydroxyl terminal groups can crosslinked, for example by reaction with polyfunctional isocyanates. Alternatively, the hydroxyl groups can be derivatized by methods known in the art to provide polyester ketal copolymers with chain-terminated reactive groups, for example (meth)acryloyl groups, isocyanate groups, or (meth)allyl ester groups. Particularly where the polyester ketal copolymers are branched, the polymers having derivatized hydroxyl groups can be crosslinked using methods known for the particular reactive groups.

Hydroxyl terminal groups can be converted to (meth)acrylic esters by reaction with (meth)acrylic acid in the presence of a suitable acid catalyst, for example sulfuric or sulfonic acid, and in the presence of one or more antioxidants and stabilizers to prevent polymerization of double bonds, or by transesterification with a (meth)acrylic ester in the presence of a suitable transesterification catalyst. The derivatized copolymers can be crosslinked (with or without a multifunctional (meth)acrylate crosslinker) or polymerized further by means of UV or radiation curing. Such (meth)acrylic ester-terminated polymers can be useful as adhesives, coatings, and thermosetting polymers for the manufacture of a broad variety of articles. The crosslinkable polymers can be processed into articles by the methods described above, and can optionally be combined with another polymer and/or a polymer additive as is known in the art.

In another specific embodiment, the polymers comprising units (3a-f) can be obtained terminated with one or more of (meth)allyl ester wherein R³ is a —CH₂CH═CH₂ or CH(CH₃)═CH₂. The end group of the polymer can be derivatized by means known in the art to provide the (meth)allyl ester, or (meth)allyl alcohol can be included in the polymerization mixture comprising at least one of hydroxy ketal ester (2a-f). In another embodiment, the polymerization mixture can comprise at least one of hydroxy ketal ester (2a-f), (meth)allyl alcohol or a reactive derivative thereof, and a comonomer having at least two terminal groups that can be hydroxyl and/or carboxyl groups, e.g., a hydroxyl-terminated linear or branched polyether. (Meth)allyl ester terminated polymers comprising fragments (3a) and/or (3b) are amenable to polymerization and useful for preparing a variety of adhesives, coatings and thermoset articles. The crosslinkable polymers can be processed into articles by the methods described above, and can optionally be combined with another polymer and/or a polymer additive as is known in the art.

In a specific embodiment, the polyester ketal polymers are copolymers obtained by co-polymerization of any of the compounds of formulae (1), specifically (1a), more specifically (1b), compounds of formulas (2a-f), or compounds of formulas (4), specifically (4a), and more specifically (4b) with one or more polyhydric alcohols having from 2 to 6 hydroxyl groups, and their reactive derivatives to produce hydroxyl-terminated ketal copolymers (copolymer polyols). The hydroxyl groups can be crosslinked or derivatized as described above. In an embodiment, copolymer polyols can be reacted with excess of one or more polyisocyanates so that substantially all hydroxyl groups of the polyol are reacted with isocyanate, to provide isocyanate-terminated ketal copolymers. The excess isocyanate is then optionally removed by distillation.

Representative polyisocyanates are of formula (11):

R⁶(—NCO)ₜ (11)

wherein t is an integer having an average value of greater than two, and R⁶ is an organic radical having a valence of t. R⁶ can be a substituted or unsubstituted hydrocarbon group (i.e., an alkane or an aromatic group) of the appropriate valency. In an embodiment, R⁶ is a group having the formula G¹-Z-G¹ wherein G¹ is a C1-12 alkylene or C6-12 arylene group and Z is —O—, —O-G²-O, —CO—, —S—, —S-G²-S—, —SO— or —SO₂—, wherein G² is a C1-12 alkylene or C6-12 arylene group. Non-limiting examples polyisocyanates of formula (11) are organic diisocyanates such as 1,4-tetramethylene diisocyanate, 1,6-hexamethylene diisocyanate, 2,2,4-trimethyl-1,6-hexamethylene diisocyanate, 1,12-dodecamethylene diisocyanate, cyclohexane-1,3- and -1,4-diisocyanate, 1-isocyanato-2-isocyanatomethyl cyclopentane, 1-isocyanato-3-isocyanatomethyl-3,5,5-trimethyl-cyclohexane (isophorone diisocyanate or IPDI), bis-(4-isocyanatocyclohexyl)methane, 2,4′-dicyclohexyl-methane diisocyanate, 1,3- and 1,4-bis-(isocyanatomethyl)-cyclohexane, bis-(4-isocyanato-3-methyl-cyclohexyl)methane, α,α,α′,α′-tetramethyl-1,3- and/or -1,4-xylylene diisocyanate, 1-isocyanato-1-methyl-4(3)-isocyanatomethyl cyclohexane, 2,4- and/or 2,6-hexahydrotoluoylene diisocyanate, 1,3- and/or 1,4-phenylene diisocyanate, 2,4- and/or 2,6-toluoylene diisocyanate, 2,4- and/or 4,4′-diphenyl-methane diisocyanate, 1,5-diisocyanato naphthalene and mixtures thereof. Polyisocyanates containing 3 or more isocyanate groups such as 4-isocyanatomethyl-1,8-octamethylene diisocyanate and aromatic polyisocyanates such as 4,4′,4″-triphenylmethane diisocyanate and polyphenyl polymethylene polyisocyanates obtained by phosgenating aniline/formaldehyde condensates can also be used.

In another aspect, organic diisocyanates include 1,6-hexamethylene diisocyanate, 1-isocyanato-3-isocyanatomethyl-3,5,5-trimethylcyclohexane (isophorone diisocyanate or IPDI), bis-(4-isocyanatocyclohexyl)methane, α,α,α′,α′-tetramethyl-1,3- and/or -1,4-xylylene diisocyanate, 1-isocyanato-1-methyl-4(3)-isocyanatomethyl cyclohexane, 2,4- and/or 2,6-hexahydrotoluoylene diisocyanate, 2,4- and/or 2,6-toluoylene diisocyanate, and 2,4- and/or 4,4′-diphenyl-methane diisocyanate.

The polyisocyanate component can be in the form of a polyisocyanate adduct, including polyisocyanate adducts containing isocyanurate, uretdione, biuret, urethane, allophanate, carbodiimide, and/or oxadiazinetrione groups. The polyisocyanates adducts have an average functionality of 2 to 6 and an NCO content of 5 to 30% by weight.

Isocyanurate group-containing polyisocyanates can be prepared as described in DE-PS 2,616,416, EP-OS 3,765, EP-OS 10,589, EP-OS 47,452, U.S. Pat. No. 4,288,586 and U.S. Pat. No. 4,324,879. The isocyanato-isocyanurates generally have an average NCO functionality of 3 to 3.5 and an NCO content of 5% to 30% by weight. In another aspect, the isocyanato-isocyanurates have an average NCO content of 10% to 25% by weight. Yet, in another aspect, the isocyanato-isocyanurates have an average NCO content of 15 to 25% by weight. Uretdione diisocyanates which can be prepared by oligomerizing a portion of the isocyanate groups of a diisocyanate in the presence of a suitable catalyst, e.g., a trialkyl phosphine catalyst, and which can be used in admixture with other aliphatic and/or cycloaliphatic polyisocyanates, particularly the isocyanurate group-containing polyisocyanates described immediately above. Biuret group-containing polyisocyanates which can be prepared according to the processes disclosed in U.S. Pat. No. 3,124,605; 3,358,010; 3,644,490; 3,862,973; 3,906,126; 3,903,127; 4,051,165; 4,147,714; or 4,220,749 by using co-reactants such as water, tertiary alcohols, primary and secondary monoamines, and primary and/or secondary diamines. These polyisocyanates can have an NCO content of 18 to 22% by weight and an average NCO functionality of 3 to 3.5. Urethane group-containing polyisocyanates which can be prepared in accordance with the process disclosed in U.S. Pat. No. 3,183,112 by reacting excess quantities of polyisocyanates, for example, diisocyanates, with low molecular weight glycols and polyols having molecular weights of less than 400, such as trimethylol propane, glycerine, 1,2-dihydroxy propane and mixtures thereof. In another aspect, the urethane group-containing polyisocyanates have a NCO content of 12 to 20% by weight and an average NCO functionality of 2.5 to 3. Allophanate group-containing polyisocyanates can be prepared according to the processes disclosed in U.S. Pat. Nos. 3,769,318, 4,160,080 and 4,177,342. Non-limiting examples of the allophanate group-containing polyisocyanates have a NCO content of 12 to 21% by weight and an average NCO functionality of 2 to 4.5. Isocyanurate and allophanate group-containing polyisocyanates can be prepared in accordance with the processes set forth in U.S. Pat. Nos. 5,124,427, 5,208,334 and 5,235,018. For example, such polyisocyanates can contain these groups in a ratio of monoisocyanurate groups to monoallophanate groups in a range of about 10:1 to 1:10. In another aspect, such polyisocyanates can contain these groups in a ratio of monoisocyanurate groups to monoallophanate groups in a range of about 5:1 to 1:7. Carbodiimide group-containing polyisocyanates can be prepared by oligomerizing di- or polyisocyanates in the presence of known carbodiimidization catalysts as described in DE-PS 1,092,007, U.S. Pat. No. 3,152,162 and DE-OS 2,504,400, 2,537,685 and 2,552,350. Polyisocyanates containing oxadiazinetrione groups can be obtained as described in U.S. Pat. No. 5,554,711.

Such isocyanate-terminated ketal copolymers have utility in the preparation of adhesives, coatings, elastomers and sealants for a wide range of industrial applications. Due to the biocompatibility of the major products formed on breakdown by acidic hydrolysis, these materials can be useful for fabrication or coating of medical devices or as the matrix materials for controlled release of pharmaceutical or agrochemical active agents. They are also useful as building blocks for the preparation of polyurethanes or polyurethane dispersions.

As described above, the polymers and copolymers described herein can be useful in a variety of applications. Accordingly, an adhesive composition comprises an adhesive formulation (e.g., a solvent, a tackifier, a crosslinking agent, and/or a polymer binder) and a polymer or copolymer comprising units of formulas (3a-f), wherein the polymer optionally comprises 2-6 terminal hydroxyl, methacryloyl, methallyl ester, or isocyanate groups. The adhesive formulation can be a hot-melt, UV-curable, radiation-curable or moisture curable adhesive, and therefore can comprise components known for use in such formulations. A coating composition comprises a coating formulation (e.g., a solvent, a filler, a pigment, a crosslinking agent, and/or a polymer binder) and a polymer or copolymer comprising units of formulas (3a-f), wherein the polymer optionally comprises 2-6 terminal hydroxyl, methacryloyl, methallyl ester, or isocyanate groups.

The polymers and copolymers described herein can be useful as thermoplastic or thermosets and used accordingly to form articles. In an embodiment, the polymers can be foamed, using mechanical frothing or chemical or physical blowing agents. The isocyanate-terminated polymers in particular can be used in the manufacture of soft or rigid polyurethane foams.

Further disclosed herein is an alternative route to compounds of formula (1), specifically (1a), more specifically (1b). In this embodiment, an oxocarboxylic acid esterified with a trimethylol (12) is condensed to form the lactone (1) as shown in Scheme (I).

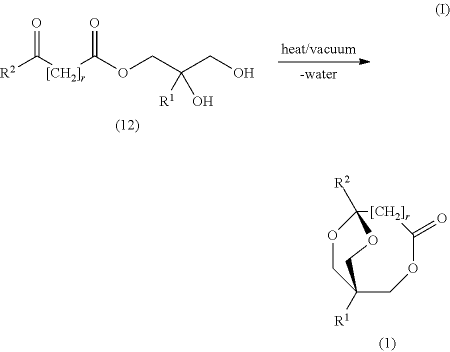

wherein r is 1-4 and each R¹ and R² is independently a C1-10 alkyl or C2-10 alkenyl. Condensation can be conducted, for example, in the presence of heat (e.g., 30-150° C.) with the removal of water, for example via a vacuum. The esterified carboxylic acid can be obtained by the acid-catalyzed decomposition of the corresponding ketal, as illustrated in the specific embodiment of Scheme II.

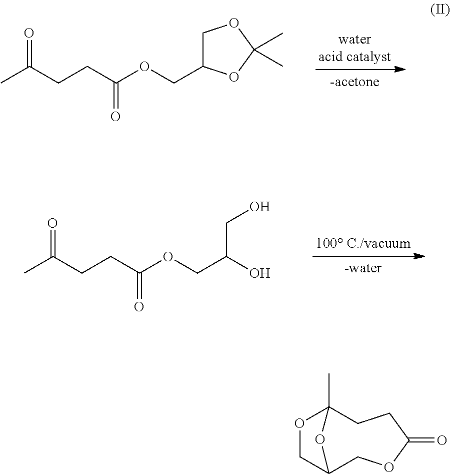

In Scheme II, solketal levulinate was treated with water in the presence of an acid catalyst to provide the diol. Heating the diol under conditions effective to remove water, e.g., a vacuum, allows ketalization of the oxo group with the diol moiety.

Compounds of formulas (1) and (4) can be used in living polymerization processes to produce polymers. In one embodiment, living polymerization can be advantageous because each of the polymer chains to begin growth at approximately the same time and at an essentially constant rate with a constant concentration of the propagating species. The end result is a polymer with a very low polydispersity index. Thus living polymerization has the advantages that it can be used to create monodisperse polymers, block copolymers, functionalized polymers, and a variety of shapes and sizes. The compounds of formulas (1) and (4) can be copolymerized in this process with other monomers such as lactide, glycolide bis-lactone, 1,4-dioxan-2-one, 1,4-dioxan-2-ones with optional C1-6alkyl or C6-12 aryl substituents at the 3-, 5-, and 6-positions, 2-oxepanone, dioxephanone monomer, e.g., 4-dioane-2-one, 1,5-dioxepan-2-one, and 4-methyl-1,5-dioxepan-2-one epsilon-caprolactone, trimethylene carbonate, dimethylene carbonate monomers, and the like. Poly(ester ethers) can be prepared by two-step ring-opening living polymerization of lactone initiated with polyether. Conditions and catalysts suitable for living polymerization are known, and include N-heterocyclic carbenes, cyclodextrins, and metal catalysts such tin(II) 2-ethylhexanoate, stannous (II)octoate, a Group 4 transition metal hydride, e.g., bis(cyclopentadienyl)zirconium(IV) chloride hydride, and various heterogeneous immobilized catalysts. The polymers or copolymers can be used, for example, in the manufacture of articles as described above.

In a specific aspect, living polymerization of the lactone compounds of formula (1) or (4) are useful in the preparation of polymers comprising of more than 90% units (2a), specifically (2c), more specifically (2e) having cis configuration and unit excess of about 100. In another aspect such polymers comprise more than 99% of units (2a), (2c), or (2e). Additionally, such polymers comprising more than 90% or 99% of units (2a), (2c), or (2e) can have average values of n in excess of about 200. Further, such polymers comprising more than 90% or 99% of units (2a), (2c), or (2e) can have average values of n in excess of about 400. Such polymers are useful thermoplastic polymers with high content of carbon derived from renewable biomass sources, and can be manufacture to be transparent.

As used herein, the singular forms “a,” “an,” and “the” are intended to include the plural forms as well, unless the context clearly indicates otherwise. The endpoints of all ranges directed to the same component or property are inclusive of the endpoint and independently combinable, except when the modifier “between” is used. The modifier “about” used in connection with a quantity is inclusive of the stated value and has the meaning dictated by the context (e.g., includes the degree of error associated with measurement of the particular quantity). A “combination” is inclusive of blends, mixtures, alloys, reaction products, and the like.

In general, the compositions or methods can alternatively comprise, consist of, or consist essentially of, any appropriate components or steps disclosed. The invention can additionally, or alternatively, be formulated so as to be devoid, or substantially free, of any components, materials, ingredients, adjuvants, or species, or steps used in the prior art compositions or that are otherwise not necessary to the achievement of the function and/or objectives of the present claims.

Unless otherwise defined, all terms (including technical and scientific terms) used have the same meaning as commonly understood by one of ordinary skill in the art to which this invention belongs. Compounds are described using standard nomenclature. Any position not substituted by any indicated group is understood to have its valency filled by a bond as indicated, or a hydrogen atom. A dash (“-”) that is not between two letters or symbols is used to indicate a point of attachment for a substituent. For example, —CHO is attached through carbon of the carbonyl group.

“Alkyl” means a straight or branched chain saturated aliphatic hydrocarbon having the specified number of carbon atoms. “Alkenyl” means a straight or branched chain aliphatic hydrocarbon having the specified number of carbon atoms and at least one carbon-carbon double bond. “Alkylene” means a straight or branched divalent aliphatic hydrocarbon group having the specified number of carbon atoms. “Aryl” means a cyclic moiety in which all ring members are carbon and a ring is aromatic. More than one ring can be present, and any additional rings can be independently aromatic, saturated or partially unsaturated, and can be fused, pendant, spirocyclic or a combination thereof. “Arylalkylene” means a group having an aryl group covalently bonded to an alkylene group bonded to both the aryl group and the position being substituted. The term “(meth)acrylate” encompasses both acrylate and methacrylate groups. The term (meth)allyl encompasses both allyl and methallyl groups.

All cited patents, patent applications, and other references are incorporated by reference in their entirety.

While embodiments of the invention have been illustrated and described, it is not intended that these embodiments illustrate and describe all possible forms of the invention. Rather, the words used in the specification are words of description rather than limitation, and it is understood that various changes can be made without departing from the spirit and scope of the invention.

1. A compound of formula (1): 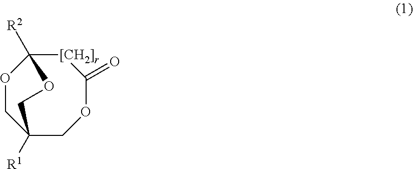
wherein
each R¹ and R² is independently a C1-10 alkyl or C2-10 alkenyl, and
r is 1-4.

2. An isolated compound of formula (1a): 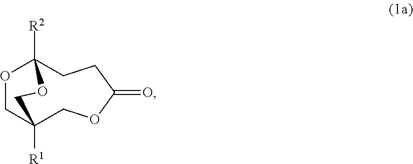
wherein each R¹ and R² is independently a C1-10 alkyl or C2-10 alkenyl.

3. An isolated compound of formula (1b): 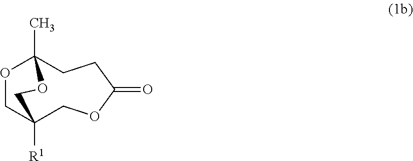
wherein R¹ is a C1-10 alkyl or C2-10 alkenyl.

4. A polymer composition comprising a polymer and a compound of claim 1.

5. An isolated compound of formula (2a): 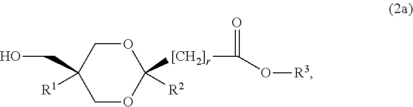
wherein
each R¹ and R² is independently a C1-10 alkyl or C2-10 alkenyl, and
R³ is C1-6 alkyl, C2-6 alkenyl, C3-6 cycloalkyl, C5-6 cycloalkenyl, C6-12 aryl, C7-C13 arylalkylene, or C2-10 alkyloxyalkylene or C3-10 alkyloxyalkyleneoxyalkylene each optionally having 1-2 two free hydroxyl groups.

6. An isolated compound of formula (2b) 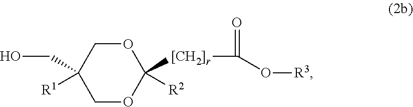
wherein
r is 1-4,
each R¹ and R² is independently a C1-10 alkyl or C2-10 alkenyl, and
R³ is C1-6 alkyl, C2-6 alkenyl, C3-6 cycloalkyl, C5-6 cycloalkenyl, C6-12 aryl, C7-C13 arylalkylene, or C2-10 alkyloxyalkylene or C3-10 alkyloxyalkyleneoxyalkylene each optionally having 1-2 two free hydroxyl groups.

7. An isolated compound of formula (2c): 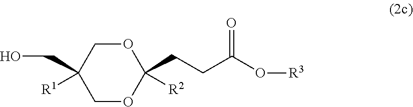
wherein
each R¹ and R² is independently a C1-10 alkyl or C2-10 alkenyl and
R³ is C1-6 alkyl, C2-6 alkenyl, C3-6 cycloalkyl, C5-6 cycloalkenyl, C6-12 aryl, C7-C13 arylalkylene, C2-10 alkyloxyalkylene, or C3-10 alkyloxyalkyleneoxyalkylene.

8. A isolated compound of formula (2d): 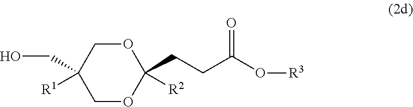
wherein
each R¹ and R² is independently a C1-10 alkyl or C2-10 alkenyl and
R³ is C1-6 alkyl, C2-6 alkenyl, C3-6 cycloalkyl, C5-6 cycloalkenyl, C6-12 aryl, C7-C13 arylalkylene, C2-10 alkyloxyalkylene, or C3-10 alkyloxyalkyleneoxyalkylene.

9. An isolated compound of formula (2e): 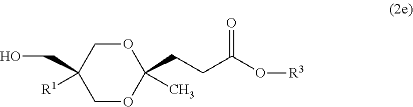
wherein
R¹ is a C1-10 alkyl, and
R³ is C1-6 alkyl, C2-6 alkenyl, C3-6 cycloalkyl, C5-6 cycloalkenyl, C6-12 aryl, C7-C13 arylalkylene, C2-10 alkyloxyalkylene, or C3-10 alkyloxyalkyleneoxyalkylene.

10. An isolated compound of formula (2f): 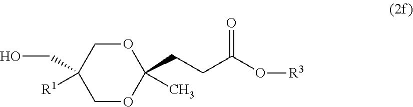
wherein
R¹ is a C1-10 alkyl, and
R³ is C1-6 alkyl, C2-6 alkenyl, C3-6 cycloalkyl, C5-6 cycloalkenyl, C6-12 aryl, C7-C13 arylalkylene, C2-10 alkyloxyalkylene, or C3-10 alkyloxyalkyleneoxyalkylene.

11. A polymer composition comprising a polymer and a compound of claim 5.

12. A compound of formula (4): 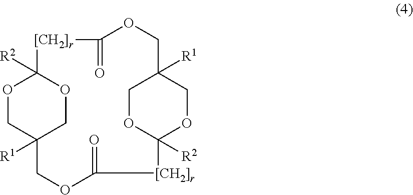
wherein
each R¹ and R² is independently a C1-10 alkyl or C2-10 alkenyl
and r=1-4.

13. A compound of formula (4a): 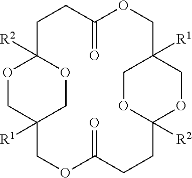
wherein each R¹ and R² is independently a C1-10 alkyl or C2-10 alkenyl.

14. A polymer composition comprising a polymer and a compound of claim 12.

15. A composition comprising
a compound of formula (1a): 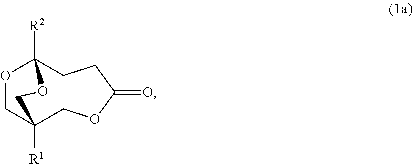
wherein each R¹ and R² is independently a C1-10 alkyl or C2-10 alkenyl; and
a compound of formula (1b): 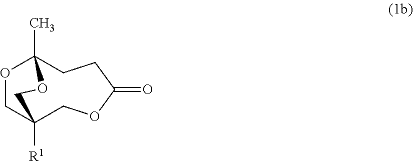
wherein R¹ is a C1-10 alkyl or C2-10 alkenyl,
wherein the ratio of the quantity of the compound of formula (1a) with respect to the quantity of the compound of formula (2b) is in a range between 0 and 0.35.

16. A composition comprising
a compound of formula (1a): 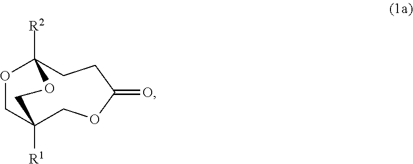
wherein each R¹ and R² is independently a C1-10 alkyl or C2-10 alkenyl; and
a compound of formula (1b): 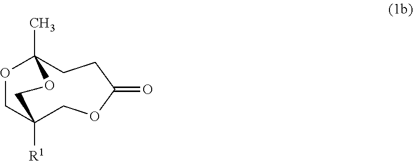
wherein R¹ is a C1-10 alkyl or C2-10 alkenyl,
wherein the ratio of the quantity of the compound of formula (1b) with respect to the quantity of the compound of formula (1a) is in a range between 0 and 0.35.

17. A polymer composition comprising a polymer and the combination of claim 15.

18. A polymer comprising a ketal unit of formula (3): 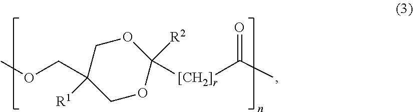
wherein
r is 1-4,
each R¹ and R² is independently a C1-10 alkyl or C2-10 alkenyl, and
n is greater than 2; and
wherein at least about 60 mol % of the units of formula (3) are units of the cis-isomer of formula (3a):

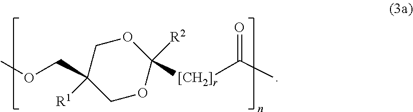

19. A polyester comprising a ketal unit of formula (3): 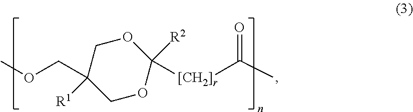
wherein
r is 1-4,
each R¹ and R² is independently a C1-10 alkyl or C2-10 alkenyl, and
n is greater than 2; and
wherein at least about 60 mol % of the units of formula (3) are units of the trans-isomer of formula (3b):

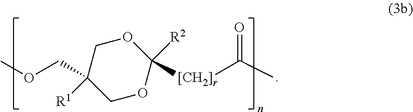

20. A polymer comprising units of formula (8): 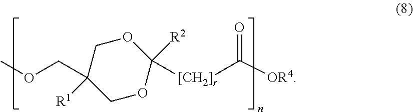
wherein
r is 1-4,
each R¹ and R² is independently a C1-10 alkyl or C2-10 alkenyl,
n is greater than 2; and
R⁴ is a C1-10 straight, branched chain, or cyclic aliphatic group, a C2-6 straight, branched chain, or cyclic aliphatic group having 1-2 double bonds, a C6-10 cyclic aromatic group, a C2-10 alkyloxyalkylene, or a C3-10 alkyloxyalkyleneoxyalkylene wherein each of the foregoing can be unsubstituted or substituted with 1-2 hydroxy groups, 1-2 (C1-3alkyl)carbonyl groups, 1-2 (C1-6)alkylcarbonyl groups, 1-2 (meth)acryloyl groups, or a combination thereof.

21. A polymer of claim 18, further comprising another polyester unit derived from a polyhydric alcohol having from 2 to 6 hydroxyl groups, a C1-36 aliphatic or C6-36 aromatic dicarboxylic or tricarboxylic acid or its reactive derivative, a hydroxylated carboxylic acid or its ester or lactone, a hydroxyl-terminated linear or branched polyether, a polyester polyol, a polyurethane polyol, a polysaccharide, a poly(3-hydroxyalkanoate), a polylactate, a polyglycolide, a poly(co-lactate/glycolate), or a combination thereof.

22. A polymer of claim 21, further comprising another ketal monomer unit derived from a hydroxy-ester or reactive derivative thereof of formula (10): 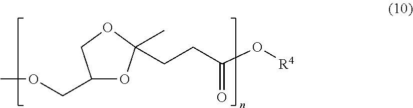
wherein r is 1-4 and R² is a C1-10 alkyl or C2-10 alkenyl.

23. A polymer of claim 18, comprising two to six of hydroxyl groups, isocyanate groups, (meth)acryloyl groups, or (meth)allyl groups.

24. A polymer of claim 18, further comprising an additional, different polymer, a polymer additive, or a combination thereof.

25. An article comprising a polymer of claim 18.

26. A foam comprising any of the polymers or compositions thereof of claim 18.

27. A method of manufacture of a compound of formula (1), comprising heating a compound of formula (3) 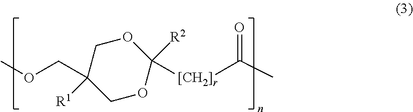
in the presence of a protonic catalyst and while removing water; and
distilling the compound of formula (1) 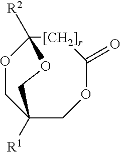
wherein
each R¹ and R² is independently a C1-10 alkyl or C2-10 alkenyl, and
r is 1-4.

28. A method of manufacture of a compound of formula (1), comprising
heating an esterified oxocarboxylic acid of formula (12). 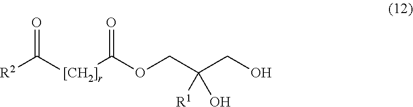
under sufficient heat with the removal of water to form the lactone of formula (1) 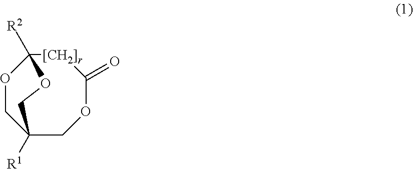
wherein
r is 1-4, and
each R¹ and R² is independently a C1-10 alkyl or C2-10 alkenyl.

29. A method of manufacture of a compound of formula (2a), comprising
transesterifying a compound of formula (1) 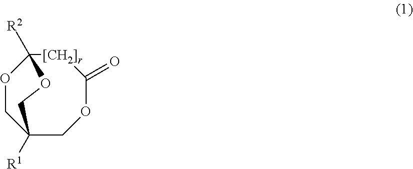
under basic conditions in the presence of a hydroxy compound of formula R³—OH to form a compound of formula (2a) 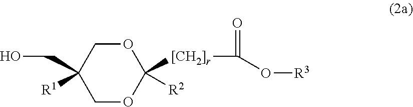
wherein
each R¹ and R² is independently a C1-10 alkyl or C2-10 alkenyl,
r is 1-4, and
R³ is C1-6 alkyl, C2-6 alkenyl, C3-6 cycloalkyl, C5-6 cycloalkenyl, C6-12 aryl, C7-C13 arylalkylene, C2-10 alkyloxyalkylene, or 3-10 alkyloxyalkyleneoxyalkylene.

30. A polymer composition obtained by a living polymerization of a compound of formula (1) or a compound of formula (4): 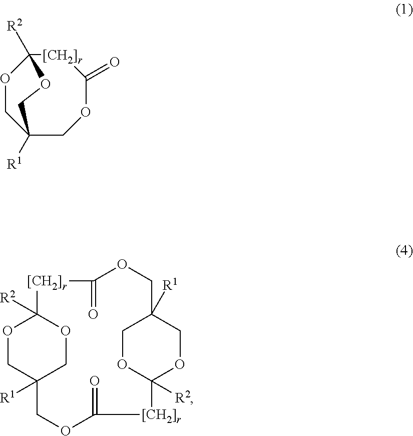
wherein
each R¹ and R² is independently a C1-10 alkyl or C2-10 alkenyl, and
r is 1-4.

31. A polymer composition of claim 30, wherein the polymer further comprises units derived from the living polymerization in the further presence of another lactone, a lactide, glycolide, bis-lactone, dioxanone, dioxepanone monomer, trimethylene carbonate, or dimethylene carbonate.

32. An article comprising the polymer of claim 30.
